(19) United States
(12) Patent Application Publication
(10) Pub. No.: US 2011/0130428 A1
(43) Pub. Date: Jun. 2, 2011

(54) Sublingual Compositions Comprising (2S) - (4E) -N-Methyl-5- (3- (5-Isopropoxypyridin) YL)-4-Penten-2-Amine

(75) Inventors: Anders X. Lindahl, Sodertalje (SE); Anders Ringberg, Sodertalje (SE)

(73) Assignee: ASTRAZENECA AB, Sodertalje (SE)

(21) Appl. No.: 12/997,274

(22) PCT Filed: Jun. 11, 2009
(86) PCT No.: PCT/SE2009/050711

§ 371 Date: Feb. 14, 2011

Related U.S. Application Data
(60) Provisional application No. 61/060,512, filed on Jun. 11, 2008.

(51) Int. Cl. A61K 31/44; A61P 25/28; A61P 25/00; A61P 25/18

(52) U.S. Cl. 514/351

(57) ABSTRACT

The present invention relates to sublingual compositions comprising (2S)-(4E)-N-methyl-5-(3-(5-iso-propoxypyridin)yl)-4-penten-2-amine or pharmaceutically acceptable salts thereof, to the preparation of said compositions and the use of thereof in therapy.

The present invention relates to sublingual compositions comprising (2S)-(4E)-N-methyl-5-(3-(5-isopropoxypyridin)yl)-4-penten-2-amine or pharmaceutically acceptable salts thereof, to the preparation of said compositions and the use thereof in therapy.

The compound (2S)-(4E)-N-methyl-5-(3-(5-isopropoxypyridin)yl)-4-penten-2-amine or pharmaceutically acceptable salts thereof, the preparation of the compound and its uses in therapy have been described in detail in U.S. Pat. No. 6,958,399 and WO2006/053082, which are hereby incorporated by reference.

The sublingual route can be used in treating patients who have difficulty in swallowing tablets, capsules or other solids, or those who have intestinal failure.

Drugs can be absorbed through mucosal surfaces, such as those in the oral cavity. Drug delivery via mucosal surfaces can be efficient because they lack the stratum corneum, a major barrier to absorption across the skin. Mucosal surfaces are also typically rich in blood supply, which can rapidly transport drugs systemically while avoiding significant degradation by first-pass hepatic metabolism.

Oral transmucosal absorption is generally rapid because of the rich vascular supply to the mucosa and the lack of a stratum corneum epidermidis. Such drug transport typically provides a rapid rise in blood concentrations, and similarly avoids the enterohepatic circulation and immediate destruction by gastric acid or partial first-pass effects of hepatic metabolism.

Because of the high permeability of the rich blood supply, the sublingual route can provide a rapid and faster onset of therapeutic action and than the oral route.

There are numerous compositions and delivery vehicles suitable for buccal or sublingual delivery of the nicotine analogs. Examples of such compositions or delivery vehicles are disclosed in U.S. Pat. Nos. 6,676,959, 6,676,931, 6,593,317, 6,552,024, 6,306,914, 6,284,264, 6,248,358, 6,210,699, 6,177,096, 6,197,331, 6,153,222, 6,126,959, 6,286,698, 6,264,981, 6,187,323, 6,173,851, 6,110,486, 5,955,098, 5,869,082, 5,985,311, 5,948,430, 5,753,256, 5,487,902, 5,470,566, 5,362,489, 5,288,498, 5,288,497, 5,269,321, 6,488,953, 6,126,959, 6,641,838, 6,576,250, 6,509,036, 6,391,335, 6,365,182, 6,280,770, 6,221,392, 6,200,604, 6,531,112, and 6,485,706, all of which are incorporated herein by reference in their entirety.

### Compound A

The compound used in the composition of the present invention is (2S)-(4E)-N-methyl-5-(3-(5-isopropoxypyridin)yl)-4-penten-2-amine or pharmaceutically acceptable salts or polymorphs thereof (hereinafter referred to as Compound A). The formula for the free base is shown below:

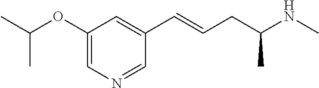

The synthesis of Compound A is described in U.S. Pat. No. 6,958,399 and WO2006/053082.
The diacyltartate salts of Compound A and the preparation thereof are described in U.S. Pat. No. 6,432,954 and in WO2002/05798.
The p-hydroxybenzoate salt of Compound A and the preparation thereof is described in WO2006/053082, which is incorporated herein by reference in its entirety.
Further salts of Compound A are described in WO2007/134038.
Polymorphic forms of Compound A are described in WO2007/134034, which is incorporated herein by reference in its entirety.

### Metabolite

A metabolite, (4E)-5-(3-(5-isopropoxypyridin)yl)-4-penten-2-amine, (hereinafter referred to as Compound B) is used in the experiments described below. The synthesis of the Compound B is described in WO 00/75110, which is incorporated herein by reference in its entirety. The formula for the free base is shown below:

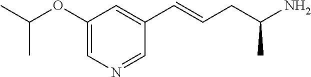

Oral administration of Compound A in the dose range from 2 to 320 mg to healthy volunteers resulted in intersubject variability in the degree of exposure to Compound A (Dunbar et al. 2006). This is probably due to the involvement of the enzymatic system CYP2D6, which is known to be subject to a genetic polymorphism, in the metabolism of Compound A (Dunbar et al. 2006; Ingelman-Sundberg et al. 2007). Dunbar and coworkers reported that a genotyping/phenotyping study has been completed and seems to confirm this hypothesis (Dunbar et al. 2006). It is thus contemplated that CYP2D6 is involved in the metabolism of Compound A.

The cytochromes P450 (CYP) constitute a superfamily of heme-thiolate proteins that catalyze the biotransformation of both endo- and xenobiotics, the latter including a wide range of prescribed pharmaceutical drugs. In humans, approximately 80% of oxidative metabolism and almost 50% of the overall elimination of commonly used drugs can be attributed to one or more of the various P450 enzymes that are classified into three families (CYP1, CYP2, and CYP3) (Wilkinson, 2005). The P450s are concentrated prominently in the liver, the principal organ of drug elimination (Lin and Lu, 2001). Therefore, hepatic CYP-mediated metabolism represents the major means by which the body eliminates drugs. In addition to the liver, the CYPs are expressed appreciably in the small intestinal mucosa, lung, kidney, brain, olfactory mucosa, and skin. Of these tissues, it has been suggested that the intestinal mucosa is the most important extrahepatic site of drug biotransformation (Lin and Lu, 2001). As a consequence, the potential exists for substantial presystemic metabolism and thus an enhanced reduction in bioavailability as the drug passes, sequentially, through the small intestine and liver. CYP2D6 is present both in the intestine and the liver (Lin and Lu, 2001; Paine et al. 2006).

CYP2D6 is the most important polymorphic enzyme active in the metabolism of pharmaceutical compounds. It is responsible for the metabolism of 25% of all pharmaceutical drugs on the market (Eichelbaum et al. 2006). This enzyme is the only one among the drug metabolizing CYPs which is not inducible, and therefore, genetic variation contributes largely to the intersubject variation in enzyme activity (Ingelman-Sundberg et al. 2007) and rate and extent of metabolism of many pharmaceutical compounds. Currently, more than 63 different functional CYP2D6 gene variants have been described and these are divided into alleles causing abolished, decreased, normal and ultrarapid enzyme activity (Ingelman-Sundberg et al. 2007).

Compound A is a drug product that belongs to Class I of the Biopharmaceutical Classification System (BCS) as it has very good solubility (highest dose strength is easily dissolved in 250 ml of aqueous media at all physiological pH values) and high intestinal permeability (more than 90% of an oral dose is absorbed). Thus, the dissolution and intestinal permeability is not the reason for the observed high intersubject variability in plasma concentrations of Compound A.

We believe that the observed high intersubject variability in systemic exposure to Compound A after oral administration is mainly due to the involvement of CYP2D6 in the metabolism during the first pass of intestinal wall and liver. Therefore, the delivery of Compound A in such a way that first pass metabolism in the intestine and liver can be avoided, should significantly reduce the intersubject variability in plasma concentrations of the drug. This would also lead to an increased bioavailability of Compound A. Alternative routes of administration that do not involve absorption from the gastrointestinal tract will by-pass first-pass metabolism in the intestine and liver include, for instance, intravenous, intramuscular or subcutaneous injection, inhalation, transdermal, intranasal, buccal and sublingual administration. The possible increase in bioavailability and/or reduced variability in plasma concentrations by sublingual administration of pharmaceutical compounds have previously been described in several patents, for instance: WO 2004/043431 or U.S. Pat. No. 5,487,898.

Sublingual administration involves the patient holding a pharmaceutical composition under their tongue while the drug dissolves in the fluid available, diffuses through the mucosa lining the mouth and from there directly into the bloodstream without passing the liver.

The oral bioavailability of Compound A in Beagle dogs has been reported to be about 30% (Gatto et al. 2004). This relatively low bioavailability is probably due to an extensive metabolism in the liver during the first-pass, as the total plasma clearance was markedly higher than the hepatic blood flow (Gatto et al. 2004). Furthermore, the dog has a buccal mucosa that is non-keratinized and has a close similarity to that of the human buccal mucosa (Shojaei 1998). Thus, this suggest that the dog is a suitable animal model for the evaluation of buccal and sublingual compositions intended for systemic delivery of Compound A.

### BRIEF DESCRIPTION OF THE FIGURES

FIG. 1 shows the plasma concentrations of Compound A and Compound B for Subject 1 following administration of Compound A both sublingually and orally.

FIG. 2 shows the plasma concentrations of Compound A and Compound B for Subject 2 following administration of Compound A both sublingually and orally.

### Preparation of the Sublingual Compositions

Compound A may be dry-mixed with one or more fillers such as mannitol or lactose, one or more disintegrant such as Polyvidon cross-linked, and optionally one or more alkaline agents such as sodium bicarbonate or magnesium hydroxide in a blender. Optionally, the dry mixture may be granulated by wet or dry granulation.

A lubricant such as sodiumstearyl-fumarate, may then be added to the mixture, followed by more blending.
Tablets can be compressed using round concave surface punches with diameters of 5 to 10 mm. Other punch shapes can be used as understood by the skilled artisan.

The preparation is performed at room temperature (16 to 25° C.). The blending times may easily be determined through routine experimentation by the person skilled in the art.

### Compositions

One embodiment of the invention relates to a composition containing:

| | | |
|---|---|---|
| Compound A or pharmaceutically acceptable salt | 1 to 50 | wt % |
| thereof | | |
| Filler | 25 to 95 | wt % |
| Disintegrant | 1 to 10 | wt % |
| Lubricant | 0.5 to 5 | wt %, and |
| Optionally Alkaline agent | 0 to 30 | wt %. |

Another embodiment of the invention relates to a composition containing:

| Compound A or pharmaceutically | 1 to 5 wt % or 10 to 15 wt % | |
|---|---|---|
| acceptable salt thereof | | |
| Filler | 50 to 95 | wt % |
| Disintegrant | 2 to 7 | wt % |
| Lubricant | 1 to 2 | wt %, and |
| Optionally Alkaline agent | 5 to 25 | wt %. |

In one embodiment the composition contains Compound A in weight % ranges of from 1 to 15%, or of from 5 to 10%, or of from 5 to 7%, or of from 10 to 15%.

The filler may be selected from calcium carbonate, dibasic calcium phosphate, tribasic calcium phosphate, calcium sulphate, kaolin, microcrystalline celluslose, powdered cellulose, sucrose, dextrose, fructose, maltose, mannose, mannitol, sorbitol, xylitol, lactitol, maltitol, lactose, erythritol, trehalose, dextrates, dextrin, maltodextrin or starch. In one embodiment the composition contains the filler in weight % ranges of from 60 to 95%, or of from 60 to 90%. In another embodiment the filler is manitol. In a further embodiment the filler is lactose.

The alkaline agent may be selected from sodium bicarbonate, magnesium hydroxide, calcium carbonate, dibasic calcium phosphate, tribasic calcium phosphate, or potassium hydroxide. In one embodiment the composition contains the alkaline agent in a weight % range of from 15 to 20%.

In another embodiment the alkaline agent is sodium bicarbonate. In one embodiment the alkaline agent sodium bicarbonate is present in weight % ranges of from 15 to 25%, or of from 15 to 20%.

In a further embodiment the alkaline agent is magnesium hydroxide. In one embodiment the alkaline agent magnesium hydroxide is present in weight % ranges of from 1 to 10%, or of from 2 to 6%.

In one embodiment the composition does not contain an alkaline agent.

The Disintegrant may be selected from polyvidon such as cross-linked polyvidon, sodium starch glycolate cross-linked carboxymethylcellulose sodium. In one embodiment the composition contains the Disintegrant in a weight % range of from 4 to 6%, or 5%. In another embodiment the Disintegrant is cross-linked polyvidon.

The lubricant may be selected from sodium stearylfumarate, magnesiumstearate calcium stearate, zinc stearate, stearic acid, talc or polyethylene glycol. In one embodiment the composition contains the lubricant in a weight % range of from 1 to 1.5%, or 1.2%. In a further embodiment the lubricant is sodium stearylfumarate.

A further embodiment of the invention relates to a composition containing:

| | |
|---|---|
| Compound A or pharmaceutically acceptable salt thereof | 1 to 20% |
| Mannitol or Lactose | 60 to 95% |
| Crosslinked polyvidon | 1 to 10% |
| Sodiumstearylfumarate | 0.5 to 5%, and |
| Optionally sodium bicarbonate or magnesium hydroxide | 0 to 25%. |

A one embodiment of the invention relates to a composition containing:

| | |
|---|---|
| Compound A or pharmaceutically acceptable salt | 5 to 10% |
| thereof | |
| Mannitol or Lactose | 60 to 95% |
| Crosslinked polyvidon | 4 to 6% |
| Sodiumstearylfumarate | 0.5 to 1.5%, and |
| Optionally sodium bicarbonate or magnesium | 0 to 20%. |
| hydroxide | |

A another embodiment of the invention relates to a composition containing:

| | |
|---|---|
| Compound A or pharmaceutically acceptable salt | 10 to 15% |
| thereof | |
| Mannitol or Lactose | 60 to 95% |
| Crosslinked polyvidon | 4 to 6% |
| Sodiumstearylfumarate | 0.5 to 1.5%, and |
| Optionally sodium bicarbonate or magnesium | 0 to 20%. |
| hydroxide | |

One embodiment of the invention relates to the use of the compositions described above for sublingual administration of Compound A.
Compound A may also be solubilized in and an aqueous media such as water or alcohol (e.g. ethanol) or a mixture of water and alcohol, and used as a sublingual solution. The mixture may comprise water and alcohol in a ratio ranging of from 99:1 to 1:75 or of from 1:75 to 1:50 or of from 1:50 to 1:10 or of from 1:10 to 1:5 or of from 1:5 to 1:1 or of from 1:1 to 1:5 or of from 1:5 to 1:10 or of from 1:10 to 1:50 or of from 1:50 to 1.75 or of from 1:75 to 1:99.
In one embodiment the composition of the invention is solubilised in an aqueous media such as water or alcohol (e.g. ethanol) or a mixture of water and alcohol and used for sublingual administration.
The solubilized Compound A may also be put in an spraying device and used as a sublingual spray.
Another embodiment relates to sublingual administration of Compound A in a spray.

### Methods of Treatment

It is advantageous that the treatment or prevention of diseases, disorders and conditions occurs without appreciable adverse side effects (e.g., significant increases in blood pressure and heart rate, significant negative effects upon the gastro-intestinal tract, and significant effects upon skeletal muscle). Compound A when employed in effective amounts, can modulate the activity of the alpha4/beta2 neuronal nicotinic receptors (α4β2 NNRs) without appreciable interaction with the NNR subtypes that characterize the human ganglia (as demonstrated by their lack of ability to elicit nicotinic function in adrenal chromaffin tissue) or skeletal muscle (as demonstrated by their lack of ability to elicit nicotinic function in cell preparations expressing muscle-type NNRs). Thus, Compound A is capable of treating and/or preventing diseases, disorders and conditions without eliciting significant side effects associated with activity at ganglionic and neuromuscular sites.

One embodiment of the invention relates to the composition of the present invention, for use in therapy.

In yet another embodiment of the present invention provides the use of the composition of the present invention, in the manufacture of a medicament for the treatment of CNS disorders.

One embodiment relates to the use of the composition of the present invention in the manufacturing of a medicament for treating or preventing disorders selected from the group consisting of Alzheimer's Disease, mild to moderate dementia of the Alzheimer's type, attention deficit disorder, attention deficit hyperactivity disorder, mild cognitive impairment, age-associated memory impairment, schizophrenia, and cognitive dysfunction in schizophrenia.

The present invention further provides a method of treating CNS disorders or disorders selected from the group consisting of age-associated memory impairment, mild cognitive impairment, pre-senile dementia (early onset Alzheimer's Disease), senile dementia (dementia of the Alzheimer's type), Lewy body dementia, HIV-dementia, vascular dementia, Alzheimer's Disease, AIDS dementia complex, attention deficit disorder, attention deficit hyperactivity disorder, schizophrenia, schizophreniform disorder and schizoaffective disorder, and cognitive dysfunction in schizophrenia, in a mammal (such as man), which comprises administering to a mammal in need of such treatment an effective Compound A in the composition of the present invention.

The composition of the invention may be given to a mammal such as a human once, twice, three or four times per 24 hours.

In one embodiment the composition of the invention is administered once per 24 hours. In another embodiment the composition of the invention is administered twice per 24 hours. In yet another embodiment the composition of the invention is administered three times per 24 hours. In a further embodiment the composition of the invention is administered four times per 24 hours.

In the context of the present specification, the term “therapy” also includes “prevention” and “prophylaxis” unless there are specific indications to the contrary. The terms “therapeutic” and “therapeutically” should be construed accordingly.

The term “Compound A” includes (2S)-(4E)-N-methyl-5-(3-(5-isopropoxypyridin)yl)-4-penten-2-amine, as well as any prodrugs thereof and pharmaceutically acceptable salts, hydrates, cocrystals and solvates of the compound and the prodrugs.

The term “disorder”, unless stated otherwise, means any condition and disease associated with activity of the α4β2 NNRs.

The term “other ingredients” means any excipients, fillers, alkaline agents, diluents, binders, lubricants, glidants, disintegrants, carriers, surfactants, flavors and mixtures thereof that are formulated with metanicotines or any prodrugs thereof and pharmaceutically acceptable salts, hydrates, and solvates thereof.

The term “appropriate period of time” or “suitable period of time” means the period of time necessary to achieve a desired effect or result. For example, a mixture can be blended until a potency distribution is reached that is within an acceptable range for a given application or use of the blended mixture.

The term “unit dose,” “unit dosage,” or “unit composition” means a physically discrete unit that contains a predetermined quantity of active ingredient calculated to produce a desired therapeutic effect. The composition can be in any suitable form for buccal, sublingual and/or intranasal administration, which forms are well known to those of skill in the art.

The term “effective amount,” as used herein means the amount determined by such considerations as are known in the art for treating or preventing central nervous system disorders, or treating addiction, inflammation or pain in an individual, wherein it must be effective to provide measurable relief in treated individuals such as exhibiting improvements including, but not limited to, more rapid recovery, improvement or elimination of symptoms or reduction of complications, or other measurements as appropriate and known to those skilled in the medical arts.

### EXAMPLES

The invention is further illustrated in following non-limiting examples.

### Tablets

Compositions of sublingual tablets comprising Compound A, 3 mg or 13 mg (dose strengths given as free base of Compound A) are shown in the tables below.

| Ingredient | Example 1 | Example 2 | Example 3 |
|---|---|---|---|
| Compound A | 4.77 mg | 4.77 mg | 4.77 mg |
| Mannitol Parteck-M200 | 65.4 mg | 57.7 mg | 61.6 mg |
| Sodium bicarbonate | — | 17 mg | — |
| Magnesium hydroxide | — | — | 3.75 mg |
| Polyvidon crosslinked | 3.75 mg | 4.25 mg | 3.75 mg |
| Sodiumstearylfumarate | 1.12 mg | 1.28 mg | 1.12 mg |
| Tablet weight | 75 mg | 85 mg | 75 mg |
| Ingredient | Example 4 | Example 5 | Example 6 |
| Compound A | 4.77 mg | 4.77 mg | 20.67 mg |
| Mannitol Parteck-M200 | — | — | 92.55 mg |
| Lactose-316 monohydrate | 65.4 mg | 57.7 mg | — |
| Sodium bicarbonate | — | 17 mg | 27 mg |
| Polyvidon crosslinked | 3.75 mg | 4.25 mg | 7.5 mg |
| Sodiumstearylfumarate | 1.12 mg | 1.28 mg | 2.25 mg |
| Tablet weight | 75 mg | 85 mg | 150 mg |

Tablets were compressed using round concave surface punches with diameter 6 mm (Example 1-5) or 8 mm (Example 6).

| Tablet characteristics | Example 1 | Example 2 | Example 3 |
|---|---|---|---|
| Tablet hardness (n = 20, N) | 66 N | 92 N | 70 N |
| Friability (n = 20, %) | 0.1% | 0.2% | 0.1% |
| Disintegration (n = 6, seconds) | 18 sec | 36 sec | 19 sec |
| Tablet characteristics | Example 4 | Example 5 | Example 6 |
| Tablet hardness (n = 20, N) | 23 N | 50 N | 65 N |
| Friability (n = 20, %) | 0.2% | 0.1% | 0.4% |
| Disintegration USP/NF (n = 6, | 15 sec | 24 sec | 16 sec |
| seconds) | | | |

| | |
|---|---|
| Dissolution (3 min) USP II, 50 rpm, phosphate buffer | 103% |
| pH6.8 (n = 6, %) | |
| Content uniformity (n = 10 mean value (mg) and | 13.38 mg 0.5% |
| r.s.d. (%)) | |

### Animal Study

This study was approved by the Ethics Committee in Gothenburg, Sweden. Two male Beagle dogs weighing 13-15 kg were used in the study. At one occasion the dogs were administered the sublingual composition under the tongue (i.e. as intended) and at the other occasion, after a washout period of 7 days, they were given the sublingual composition orally (i.e. into the stomach by gavage together with 40 ml of water). The dose was 13 mg (as base) of Compound A (hydroxybenzoate salt) at both study occasions (Example 6 in Tables above).

The dogs were fasted overnight and until four hours after dose administration. Water was allowed ad lib until one hour before dosing and after three hours following dosing. To minimize the chance of swallowing during the sublingual study occasion, the dogs were anesthetized by intravenous bolus injection of Diprivan (10 mg/ml, AstraZeneca, Sweden) 0.8-1 ml/kg, followed by intubation, connection to a respiratory ventilator (Servo ventilator 900C, Siemens-Elema AB, Sweden) and capnograph (RespSense, MedAir AB, Sweden) and given additional anesthesia with 1.5% Isofluran (Isoba Vet., Schering-Plough AB, Sweden). The dogs were kept anesthetized for one hour after the administration of the sublingual composition under the tongue. The dogs were not anesthetized during the oral administration occasion. Anesthetized dogs have previously been used when studying sublingual compositions (Qiu et al. 1999).

Venous blood samples (2 mL) were collected before (0) and after 5, 15, 30, 45 minutes, 1.0, 1.5, 2.0, 3.0, 4.0, 6.0 and 8.0 hours after dosing. The samples were collected into vacuum tubes (BD Vacutainer K2-EDTA, Becton&Dickinson AB, Sweden), placed on ice and centrifuged for 10 minutes (4° C., 1500 g) within 30 minutes after sampling. The plasma was then transferred to 1.8 ml Nunc Cryo tubes (InterMed A/S, Denmark) and frozen (−20° C.) pending analysis.

The plasma samples (50 μL) were analyzed for Compound A and its metabolite Compound B by a standard LC-MS/MS analytical method. The determinations of the concentration of Compound A and Compound B in plasma was performed by liquid-liquid extraction followed by reversed-phase liquid chromatography and tandem mass spectrometry. Determinations of both Compound A and Compound B were performed in the range 0.8 to 200 nmol/L. Samples outside the range of determination were diluted up to 10 times. Quality control samples at three concentrations were interspersed with the study samples at 2.40, 16.0 and 160 nmol/L. The internal standard used in the assay was Deuterium labeled Compound A (AstraZeneca R&D, Sweden). The limit of quantification, LOQ was 0.8 nmol/L for both Compound A and Compound B. Quality Control procedures followed were based upon the Guidance for Industry, Bioanalytical Methods Validation, U.S. Department of Health and Human Services, Food and Drug Administration, Center for Drug Evaluation and Research (CDER), Center for Veterinary Medicine (CVM), May 2001, BP.

Exposure of animals to Compound A and it's metabolite Compound B expressed as area under the curve from time zero to the last sampling time, AUC(0-8 h), was obtained by means of non compartmental analysis in WinNonlin (version 4.0, Pharsight Corporation, USA). The maximum concentration, Cₘₐₓ, and time of maximum concentration, Tₘₐₓ, were direct observations from the plasma concentration versus time data. AUC(0-8 h) was calculated by means of the linear up-log down method. The area under the curve from zero to infinity, AUC, was calculated in a similar manner, with extrapolation from time of the last observed concentration, tₗₐₛₜ, to infinity by adding the ratio of C_{pred} (predicted plasma concentration at tₗₐₛₜ) and terminal slope factor. The terminal slope factor and subsequently the terminal half life (T_{1/2}) were estimated in WinNonlin by log linear regression of the terminal phase (4 data points were used) of the plasma concentration time curve. Actual time points were used in the non compartmental analysis. Observations below the LOQ were treated as missing values in the analysis. The difference in exposure (Cₘₐₓ, AUC 0-8 h) following sublingual administration compared to oral administration was calculated as (%): 100*sublingual value/oral value.

### Results

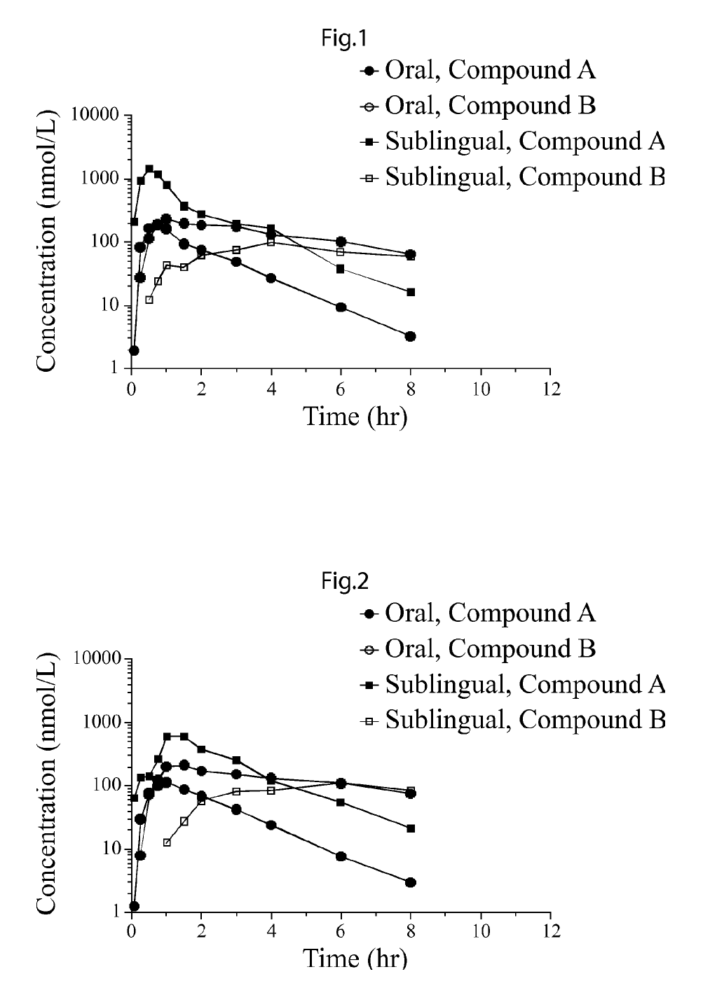

FIGS. 1 and 2 show the plasma concentrations of Compound A and Compound B, for Subjects 1 and 2 respectively, following administration of Compound A sublingually and orally.

Table 1 collects the calculated pharmacokinetic parameters for Compound A and the metabolite Compound B after the administration of the Compound A solid composition sublingually and orally, respectively. Both the Cₘₐₓ and AUC of Compound A were much higher following sublingual administration compared to oral administration. Although the Tₘₐₓ for Compound A was not significantly shorter after sublingual administration (0.5 and 1 hour) than after oral administration (0.75 and 1 hours), the plasma concentrations of Compound A were much higher following sublingual administration compared to oral administration. Already at 5 minutes after sublingual administration the plasma concentration of Compound A was about 200 and 60 nmol/L for Subject 1 and 2, respectively. This can be compared to about 2 and 1.5 nmol/L following oral administration to Subject 1 and 2, respectively (FIGS. 1 and 2). This shows that sublingual delivery of Compound A will result in a faster appearance and higher plasma concentrations of the compound in the systemic blood stream, especially during the first hour after administration, compared to conventional oral delivery where the composition is swallowed. This may result in a faster onset of pharmacological effects of the compound in patients.

TABLE 1
Pharmacokinetic parameters for Compound A and the metabolite Compound B after the
administration of the Compound A solid composition sublingually and orally to two dogs
(subject 1 and 2).

| | | | Tmax | Cmax | AUC | T½ | AUC (0-8 h) |
|---|---|---|---|---|---|---|---|
| Treatment | Subject | Analyte | (h) | (nmol/L) | (h * nmol/L) | (hr) | (h * nmol/L) |
| Oral | 1 | Compound A | 0.75 | 190 | 380 | 1.3 | 370 |
| | 1 | Compound B | 1 | 230 | NC | NC | 1000 |
| | 2 | Compound A | 1 | 120 | 290 | 1.3 | 280 |
| | 2 | Compound B | 1.5 | 210 | NC | NC | 1000 |
| Sublingual | 1 | Compound A | 0.5 | 1400 | 2100 | 1.3 | 2000 |
| | 1 | Compound B | 4 | 99 | NC | NC | 510 |
| | 2 | Compound A | 1 | 610 | 1500 | 1.4 | 1500 |
| | 2 | Compound B | 6 | 110 | NC | NC | 590 |

NC not calculated

Table 2 shows the comparison in exposure (Cₘₐₓ and AUC) of Compound A and the metabolite Compound B after sublingual and oral administration of the solid composition of Compound A, respectively. The Cₘₐₓ increased by 740% and 510% following sublingual administration compared to oral administration for Subject 1 and 2, respectively (Table 2). The AUC increased by 550 and 520% for Subject 1 and 2, respectively, compared to oral administration (Table 2). The exposure (Cₘₐₓ, AUC 0-8 h) of the metabolite Compound B decreased by about 30-70% following sublingual administration of Compound A compared to following oral administration (Table 2).

TABLE 2
The relative difference in exposure of Compound A and Compound B
after sublingual administration of Compound A compared to the
corresponding exposure following oral administration of Compound
A to two dogs (Subject 1 and 2).

| | |
|---|---|
| AUC^{a} | Cmax |

| | | |
|---|---|---|
| Subject | Analyte | (% difference) |

| | | | |
|---|---|---|---|
| 1 | Compound A | 550 | 740 |
| 2 | Compound A | 520 | 510 |
| 1 | Compound B | 51 | 43 |
| 2 | Compound B | 59 | 52 |

^{a}AUC for Compound A and AUC (0-8 h) for the metabolite Compound B.

The same dose strength of Compound A may be administered sublingually to patients regardless of whether the patient is an extensive or poor metaboliser of Compound A, i.e., regardless of the patients genotype of CYP2D6 or other genetically polymorphic enzymes present in the gut wall and/or liver.

The results above show that sublingual administration results in less systemic exposure of metabolites to Compound A that are formed during the first pass through the gut wall and liver.

Sublingual delivery of Compound A results in a higher bioavailability compared to conventional oral administration where the composition is swallowed and the compound will pass through the intestinal wall and liver before the systemic circulation is reached. This means that the dose can be decreased and still result in the same or similar plasma concentrations of Compound A, i.e. the same pharmacological response can be achieved with a lower dose.

Sublingual delivery of Compound A will decrease the variability in both rate (e.g. Cₘₐₓ) and extent (e.g. AUC) of bioavailability between patients, especially between patients belonging to different CYP2D6 genotypes.

Sublingual delivery of Compound A will result in a much faster appearance of the compound in the systemic blood stream compared to conventional oral delivery where the composition is swallowed. This may result in a faster onset of pharmacological effects of the compound in patients.

One embodiment of the invention relates to a method for increasing the Cₘₐₓ by at least 50, 100, 150, 200 or 300%, preferably more than 400%, compared to oral administration, following sublingual administration of Compound A in a sublingual composition of the present invention as described above.

Another embodiment of the invention relates to a method for increasing the AUC by at least 20, 50, 100, 150, 200 or 300%, preferably more than 400%, compared to oral administration, following sublingual administration of Compound A in a sublingual composition of the present invention as described above.

Yet a further embodiment of the invention relates to a method for decreasing the exposure (Cₘₐₓ, AUC 0-8 h) of metabolite Compound B by about 40 to 60%, compared to oral administration, following sublingual administration of Compound A in a sublingual composition of the present invention as described above.

### REFERENCES

Dunbar G, Demazières A, Monreal A, Cisterni C, Metzger D, Kuchibhatla R and Luthringer R. Pharmacokinetics and safety profile of ispronicline (TC-1734), a new brain nicotinic receptor partial agonist, in young healthy male volunteers. J Clin Pharmacol. 2006; 46:715-726.
Ingelman-Sundberg M, Sim S C, Gomez A, Rodriguez-Antona C. Influence of cytochrome P450 polymorphisms on drug therapies: Pharmacogenetic, pharmacoepigenetic and clinical aspects. Pharmacology & Therapeutics 2007; 116:496-526 Eichelbaum M, Ingelman-Sundberg M. and Evans W.E. Pharmacogenomics and individualized drug therapy. Annu Rev Med 2006; 57:119-137.
Lin J H, Lu A Y. Interindividual variability in inhibition and induction of cytochrome P450 enzymes. Annu Rev Pharmacol Toxicol. 2001; 41:535-567.
Wilkinson G R. Drug metabolism and variability among patients in drug response. N Engl J Med 2005; 352: 2211-2221
Paine M., Hart H., Ludington S., Haining R., Rettie A., and Zeldin D. The human intestinal cytochrome P450 “pie”. Drug Metabolism and Disposition 2006; 34:880-886.
Gatto G J, Bohme G A, Caldwell W S, Letchworth S R, Traina V M, Obinu M C, Laville M, Reibaud M, Pradier L, Dunbar G, Bencherif M. Compound A: an orally active neuronal nicotinic acetylcholine receptor modulator with antidepressant, neuroprotective and long-lasting cognitive effects. CNS Drug Rev. 2004 Jan. 1; 10(2):147-66.
Shojaei A. Buccal mucosa as a route for systemic drug delivery: A review. J Pharm Pharmaceut Sci. 1998; 1:15-30.
Qui Y, Johnson H W, Reiland T H and Fu Lu M-Y. Sublingual absorption of leuprolide: comparison between human and animal models. Int J. Pharm. 1999; 179:27-36.

1-9. (canceled)

10. A composition comprising:
about 1 to 50 weight % of (2S)-(4E)-N-methyl-5-(3-(5-isopropoxypyridin)yl)-4-penten-2-amine or a pharmaceutically acceptable salt thereof;
about 25 to 95 weight % of a filler;
about 1 to 10 weight % of a disintegrant;
about 0.5 to 5 weight %, of a lubricant, and optionally,
about 0 to 30 weight % of an alkaline agent.

11. A composition according to claim 1, comprising:
about 1 to 20 weight % of (2S)-(4E)-N-methyl-5-(3-(5-isopropoxypyridin)yl)-4-penten-2-amine or pharmaceutically acceptable salt thereof;
about 60 to 95 weight % of a filler that is mannitol or lactose;
about 1 to 10 weight % of a disintegrant that is crosslinked polyvidon;
about 0.5 to 5 weight % of a lubricant that is sodiumstearylfumarate, and optionally,
about 0 to 25 weight % of an alkaline agent that is sodium bicarbonate or magnesium hydroxide.

12. A method of treating CNS disorders or disorders selected from the group consisting of: age-associated memory impairment, mild cognitive impairment, pre-senile dementia, early-onset Alzheimer's Disease, senile dementia, dementia of the Alzheimer's type, Lewy body dementia, HIV-dementia, vascular dementia, Alzheimer's Disease, AIDS dementia complex, attention deficit disorder, attention deficit hyperactivity disorder, schizophrenia, schizophreniform disorder, schizoaffective disorder, and cognitive dysfunction in schizophrenia, the method comprising administering to a patient in need of such treatment an effective amount of a composition according to claim 10.

13. A method of treating CNS disorders or disorders selected from the group consisting of: age-associated memory impairment, mild cognitive impairment, pre-senile dementia, early-onset Alzheimer's Disease, senile dementia, dementia of the Alzheimer's type, Lewy body dementia, HIV-dementia, vascular dementia, Alzheimer's Disease, AIDS dementia complex, attention deficit disorder, attention deficit hyperactivity disorder, schizophrenia, schizophreniform disorder, schizoaffective disorder, and cognitive dysfunction in schizophrenia, the method comprising administering to a patient in need of such treatment an effective amount of a composition according to claim 11.

14. A method for achieving a Cₘₐₓ at least 50, 100, 150, 200, 300 or 400% greater than that achieved by oral administration of (2S)-(4E)-N-methyl-5-(3-(5-isopropoxypyridin)yl)-4-penten-2-amine, comprising sublingual administration of a composition according to claim 10.

15. A method for achieving a Cₘₐₓ at least 50, 100, 150, 200, 300 or 400% greater than that achieved by oral administration of (2S)-(4E)-N-methyl-5-(3-(5-isopropoxypyridin)yl)-4-penten-2-amine, comprising sublingual administration of a composition according to claim 11.

16. A method for achieving an AUC of at least 50, 100, 150, 200, 300 or 400% greater than that achieved by oral administration of (2S)-(4E)-N-methyl-5-(3-(5-isopropoxypyridin)yl)-4-penten-2-amine, comprising sublingual administration of a composition according to claim 10.

17. A method for achieving an AUC of at least 50, 100, 150, 200, 300 or 400% greater than that achieved by oral administration of (2S)-(4E)-N-methyl-5-(3-(5-isopropoxypyridin)yl)-4-penten-2-amine, comprising sublingual administration of a composition according to claim 11.

18. A method for decreasing the exposure (C., AUC 0-8 h) to a metabolite (4E)-5-(3-(5-isopropoxypyridin)yl)-4-penten-2-amine by about 40 to 60%, as compared to oral administration, by sublingual administration of (2S)-(4E)-N-methyl-5-(3-(5-isopropoxypyridin)yl)-4-penten-2-amine or a pharmaceutically acceptable salt thereof in a composition according to claim 1.

19. A method for decreasing the exposure (C., AUC 0-8 h) to a metabolite (4E)-5-(3-(5-isopropoxypyridin)yl)-4-penten-2-amine by about 40 to 60%, as compared to oral administration, by sublingual administration of (2S)-(4E)-N-methyl-5-(3-(5-isopropoxypyridin)yl)-4-penten-2-amine or a pharmaceutically acceptable salt thereof in a composition according to claim 2.
